(19) United States
(12) Patent Application Publication
(10) Pub. No.: US 2016/0184113 A1
(43) Pub. Date: Jun. 30, 2016

(54) LIMB SOCKET LINER WITH SEAL

(71) Applicant: Wayne A. KONIUK, San Francisco, CA (US)

(72) Inventor: Wayne A. KONIUK, San Francisco, CA (US)

(21) Appl. No.: 14/334,537

(22) Filed: Jul. 17, 2014

Related U.S. Application Data
(60) Provisional application No. 61/955,480, filed on Mar. 19, 2014.

(51) Int. Cl. A61F 2/80

(52) U.S. Cl. CPC A61F 2/80; A61F 2002/802

(57) ABSTRACT

A system for attaching a prosthetic limb to a residual limb. In embodiments, the system includes a liner having an elastic sleeve adapted to accommodate a residual limb; and a socket adapted to surround the elastic sleeve, the socket having a vacuum port, an inner surface, and a vacuum channel extending radially outward from the inner surface and longitudinally within the socket, the vacuum channel being configured so as to be in fluid communication with the vacuum port when the residual limb and liner are disposed in the socket. In some embodiments the sleeve has an annular groove disposed on an inner surface of the sleeve and a flexible annular seal extending radially inward from an inner surface of the sleeve below the groove, the annular groove being adapted receive at least part of the annular seal when the annular seal is moved toward the interior surface of the sleeve.

### CROSS REFERENCE TO RELATED APPLICATIONS

This application claims the benefit under 35 U.S.C. §119 of U.S. patent application Ser. No. 61/955,480, filed Mar. 19, 2014, which is herein incorporated by reference in its entirety.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND

Prosthetic legs are often attached to the residual limb with a socket. Vacuum may be applied to the inside of the socket to hold it in place. In some cases, a liner may be added between the socket and the patient's skin for comfort. Examples of prior prosthetic leg attachment mechanisms may be found in U.S. Pat. No. 7,025,793; U.S. Pat. No. 7,144,429; U.S. Pat. No. 8,409,299; and US Publ. No. 2013/0197670.

### SUMMARY OF THE DISCLOSURE

A common drawback of existing liners for vacuum-based prosthetic leg attachment mechanisms is the potential for loss of vacuum, and detachment of the prosthesis, when the patient moves. This problem is particularly acute for prostheses attached below the knee and extending above the knee, where bunching of the liner material when the knee is bent can put too much pressure on the patient's tissue in some spots and can lead to loss of vacuum between the limb and the socket.

The invention relates to an improved liner for use with a prosthetic limb. In particular, the invention relates to a liner for use with the socket of a prosthesis attached to a residual limb. The invention also relates to a socket with a vacuum channel to communicate vacuum longitudinally into the socket.

In one aspect of the invention, the invention is a liner that includes an elastic sleeve adapted to accommodate a residual limb, the elastic sleeve having an open end and a closed end; an annular groove disposed on an inner surface of the sleeve; and a flexible annular seal extending radially inward from an inner surface of the sleeve below the groove, the annular groove being adapted receive at least part of the annular seal when the annular seal is moved toward the interior surface of the sleeve. The liner may also include an annular element on an exterior surface of the sleeve and adapted to engage an interior surface of a limb prosthesis socket. In some embodiments, the liner includes a vacuum port disposed in the closed end of the sleeve and configured to communicate a vacuum source with the interior of the sleeve.

Another aspect of the invention provides a method of attaching a prosthetic limb to a residual limb of a patient, including the following steps: covering a portion of the residual limb with a liner, the liner having a flexible seal extending radially inward from an inner surface of the sleeve; engaging the seal with skin of the residual limb; and applying a prosthetic limb socket over the sleeve. Some embodiments of the invention also include the step of everting the sleeve prior to the covering step. Some embodiments also include the step of moving the seal into a groove disposed on the inner surface of the sleeve. Some embodiments add the step of applying vacuum in a space within the liner below the seal.

Yet another aspect of the invention provides a system for attaching a prosthetic limb to a residual limb, the system including: a liner having an elastic sleeve adapted to accommodate a residual limb; and a socket adapted to surround the elastic sleeve, the socket having a vacuum port, an inner surface, and a vacuum channel extending radially outward from the inner surface and longitudinally within the socket, the vacuum channel being configured so as to be in fluid communication with the vacuum port when the residual limb and liner are disposed in the socket. In some embodiments, the vacuum channel is disposed in the socket. The system may also have a plurality of vacuum channels extending radially outward from the inner surface and configured so as to be in fluid communication with the vacuum port when the residual limb and liner are disposed in the socket. These vacuum channels may be disposed radially around the socket.

In some embodiments, the sleeve has an annular groove disposed on an inner surface of the sleeve and a flexible annular seal extending radially inward from an inner surface of the sleeve below the groove, the annular groove being adapted receive at least part of the annular seal when the annular seal is moved toward the interior surface of the sleeve. The sleeve may also have an annular element on an exterior surface of the sleeve that is adapted to engage an interior surface of the socket. The sleeve may also have a vacuum port disposed in the closed end of the sleeve and configured to communicate vacuum from the socket vacuum port with the interior of the sleeve.

Still another aspect of the invention provides a method of attaching a prosthetic limb to a residual limb of a patient. The method may include the steps of: covering a portion of the residual limb with a liner; mounting a prosthetic limb socket over the sleeve; applying vacuum to a space between a closed end of the socket and the sleeve; and communicating the vacuum from the space to a vacuum channel extending longitudinally from the space and radially outward from an interior surface of the socket. In some embodiments, the method also includes the step of applying the vacuum to a space within the liner. In some such embodiments, the liner may have a flexible seal extending radially inward from an inner surface of a sleeve, the method further comprising applying the vacuum to the space within the liner below the seal.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 is perspective view of a liner for a prosthetic limb according to the invention.

FIG. 2 is a cross-sectional view of the liner shown in FIG. 1 mounted on a residual limb.

FIG. 3 is a perspective view of a socket for a prosthetic limb according to one aspect of the invention.

FIG. 4 a cross-sectional view of the socket of FIG. 3 taken along line A-A in FIG. 3.

FIG. 5 is a side elevational view of a form used in a method of making the socket of FIG. 3.

FIG. 6 is a perspective view of a liner that may be used with the socket of FIG. 3.

FIG. 7 is a perspective view of a tool that may be used in a method of making the socket of FIG. 3.

FIG. 8 illustrates the step of mounting the liner of FIG. 6 onto the form of FIG. 5 pursuant to a method of making the socket of FIG. 3.

FIG. 9 is a perspective view showing the application of the tool of FIG. 7 to the liner and form of FIG. 8 pursuant to a method of making the socket of FIG. 3.

FIG. 10 is a perspective view showing the socket of FIG. 3 formed over the liner and form of FIG. 8.

FIG. 11 is a cross-sectional view of the socket, liner, tool and form of FIG. 10 along the lines A-A of FIG. 10.

### DETAILED DESCRIPTION

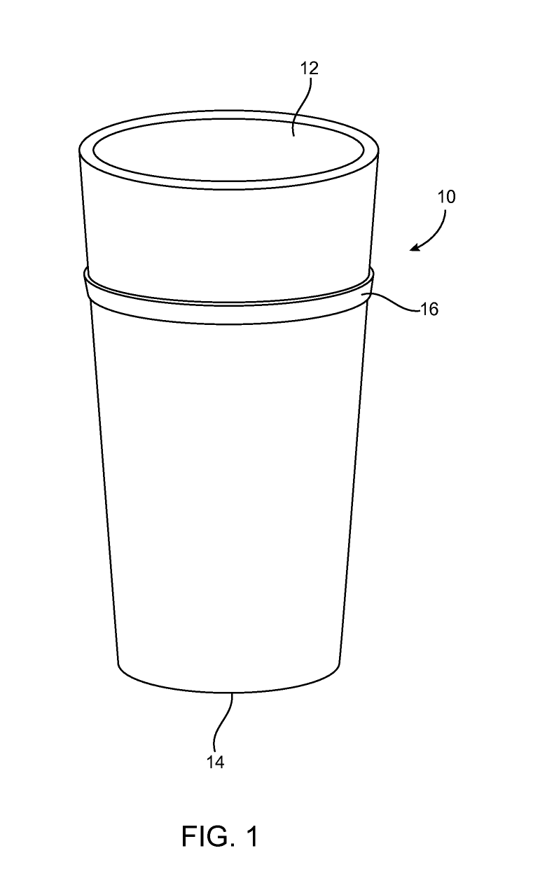

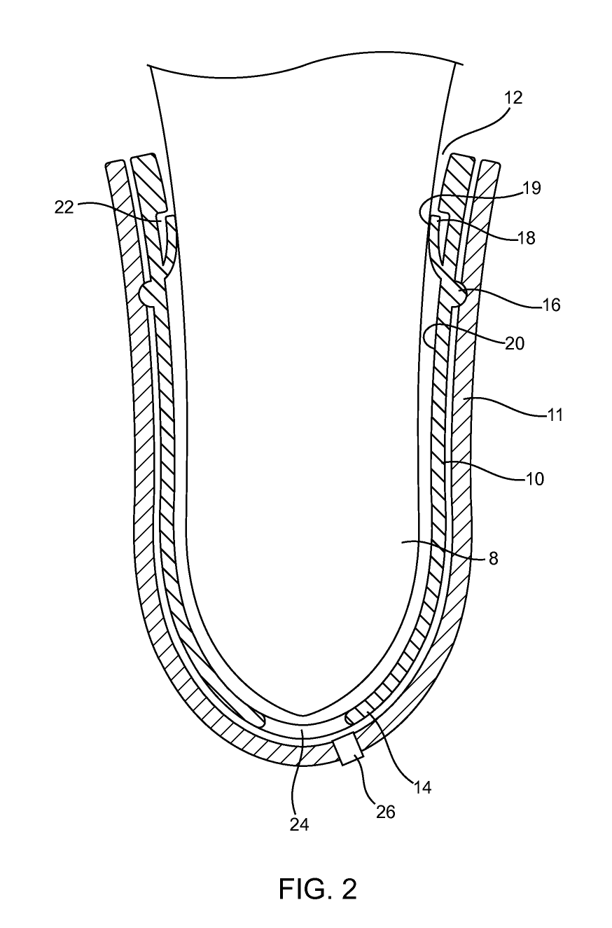

FIGS. 1-2 show a liner 10 for prosthetic limb 8. Liner 10 is formed from a flexible material, such as silicone, urethane, thermoplastic elastomer (or any other suitable material). While an elastomeric material will adapted to specific dimensions of the patient's residual limb (e.g., an arm or a leg), the sleeve may optionally be shaped to match the contours of the patient's residual limb. Liner 10 has an opening 12 at the top and a closed bottom end 14. An annular ring 16 on the outside of liner 10 is configured to engage with the inside surface of the socket 11 of a prosthetic limb (not shown). Ring 16 may have a rectangular cross-sectional shape (as shown in FIG. 1), a more rounded cross-sectional shape (as shown in FIG. 2), or any other suitable shape.

A flexible seal 18 extends radially inward and axially upward from an inner surface 20 of liner 10. Seal 18 may be formed from the same material as the liner or from any suitable elastomeric material. A groove 22 extends radially into the inner surface 20 of liner 10. Groove 22 and seal 18 each has a size, shape and location that enable seal 18 to move into groove 22 as seal 18 is stretched upward and radially outward.

An optional vacuum port 24 is shown at the bottom of liner 10. If vacuum is applied to the inside of a socket of a prosthetic limb (e.g., through port 26 in socket 11), vacuum port 24 communicates the vacuum to the interior of sleeve 10. Vacuum port 24 may be located at any position below seal 18. Vacuum port 24 may be, e.g., a slit formed in the elastomeric material of the sleeve.

In use, liner 10 may be everted, then rolled onto the patient's residual limb 8 so that opening 12 surrounds the limb 8 and the closed end 14 covers the distal end of the residual limb 8. After mounting the liner on the residual limb 8, seal 18 extends upward from the portion of the inner surface of liner 10 to which seal 18 is attached, and the edge 19 of seal 18 rests against the patient's skin. While this method of rolling the sleeve onto the limb will naturally create a vacuum between the sleeve and the limb, additional vacuum may be applied through optional port 24 to pull the seal 18 further downward and inward against the patient's skin, thereby tightening the attachment of liner 18 to the patient. Vacuum applied to port 24 can also help remove sweat from the space between limb 8 and liner 10.

In addition, the optional anatomically contoured shape of liner 10 can help ensure a close fit between liner 10 and the patient's limb. This close fit helps maintain the vacuum and helps keep the prosthesis securely attached to the residual limb. The groove 22 also helps maintain the vacuum by giving the seal a place to go as the patient moves the limb and changes the shape of the resilient sleeve.

The liner of this invention provides a more secure attachment of a prosthetic limb to a patient. In some embodiments, the attachment is strong enough that the liner does not even need to extend, e.g., over a knee or other joint, thereby removing one potential cause of vacuum failure.

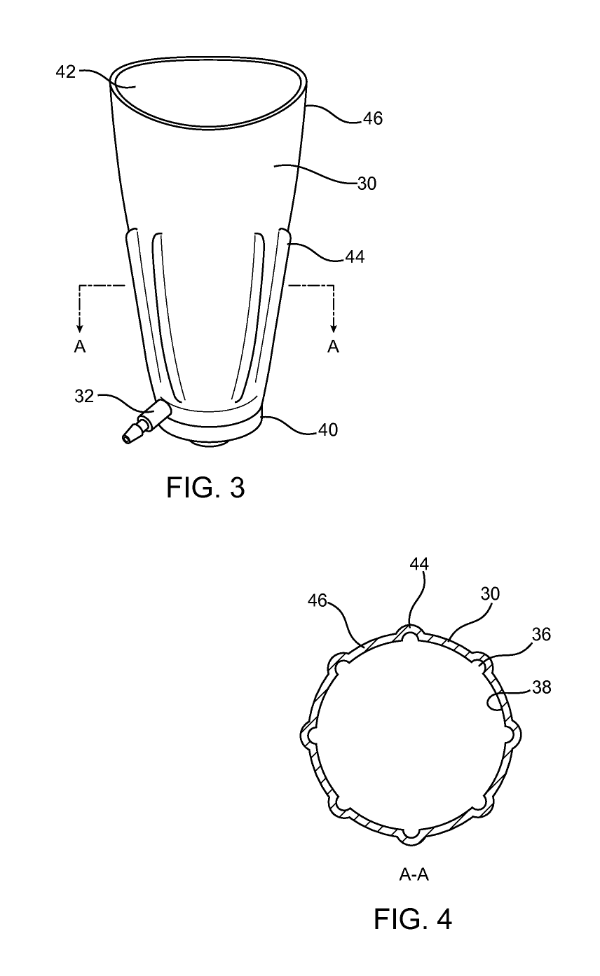

FIGS. 3 and 4 show an alternative socket 30 for use in attaching a prosthetic limb to a patient's residual limb. Socket 30 may be used with a liner (not shown in FIGS. 3 and 4), such as the liner 10 described above. As shown in FIG. 3, a vacuum port 32 in socket 30 may be used to apply vacuum to the interior of socket 30 to provide an attachment force between the prosthetic limb and the patient's residual limb in a manner known in the art. Vacuum port may have a one-way valve 34. A second port (not shown) may be provided at the closed end of the socket with a one-way valve to provide a way for air to be pushed out of the socket as it is mounted on the patient's residual limb.

Socket 30 has a plurality of vacuum channels 36 extending radially outward from an interior surface 38 of socket 30. Vacuum channels 36 extend longitudinally from the closed end 40 of socket 30 toward, but not all the way to, the open end 42 of socket 30. Vacuum channels enable fluid communication between the vacuum port 32 and the interior of socket 30 to expand the surface area over which the vacuum applied to the interior of the socket acts to provide the holding force between the socket and the patient's residual limb. While FIGS. 3 and 4 show ridges 44 on the exterior surface 46 of socket 30 corresponding to the vacuum channels, these ridges are optional. The exterior surface of the socket may be smooth in other embodiments.

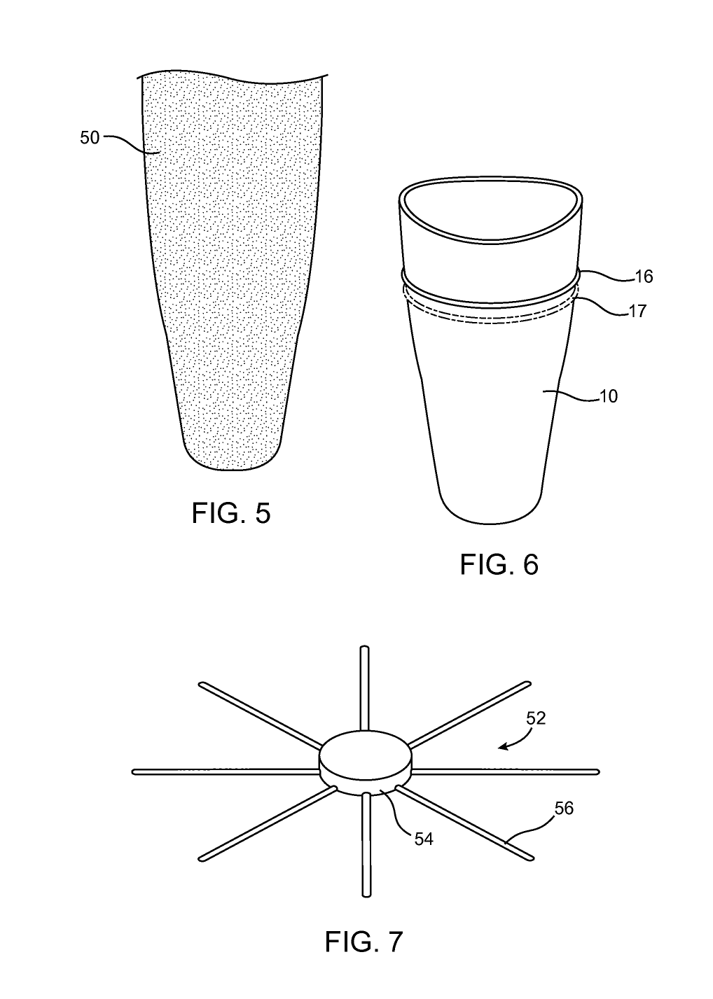

FIGS. 5-11 show a method of making the socket 30 shown in FIGS. 3 and 4. FIG. 5 shows a form 50 that is a positive representation of the patient's residual limb. Form 50 may be made from a cast or scan of the residual limb which is then turned into a positive representation of the residual limb segment. Form 50 may be plaster, rigid foam, or any other suitable material.

FIG. 6 shows a liner 10, such as the liner described above with respect to FIGS. 1 and 2. Liner 10 may have a single exterior ring 16, as shown in FIGS. 1 and 2. Liner 10 may optionally have multiple exterior rings, such as the additional ring 17 shown in phantom in FIG. 6.

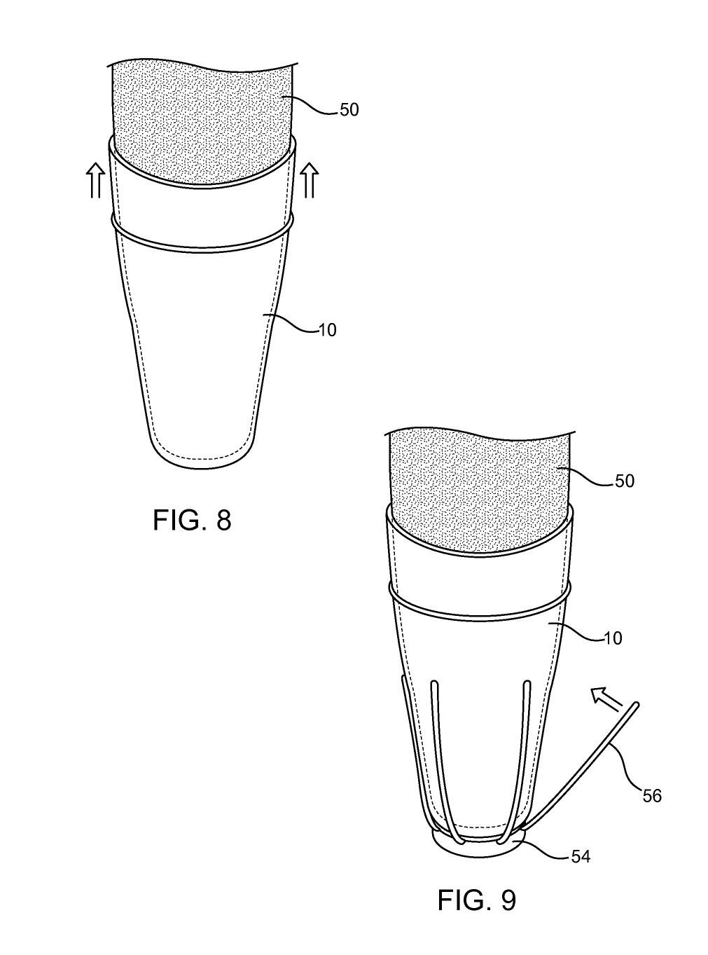

To make socket 30, liner 10 is placed over form 50, as shown in FIG. 8. A channel shaping tool 52 is then applied to the base of liner 10, as shown in FIG. 9. Tool 52 has a central base 54 a plurality of arms 56 extending from base 52. Each arm 56 corresponds to a vacuum channel to be formed in socket 30. In the illustrated embodiment, arms 56 are flexible and can adapt to the shape of liner 10 and/or form 50.

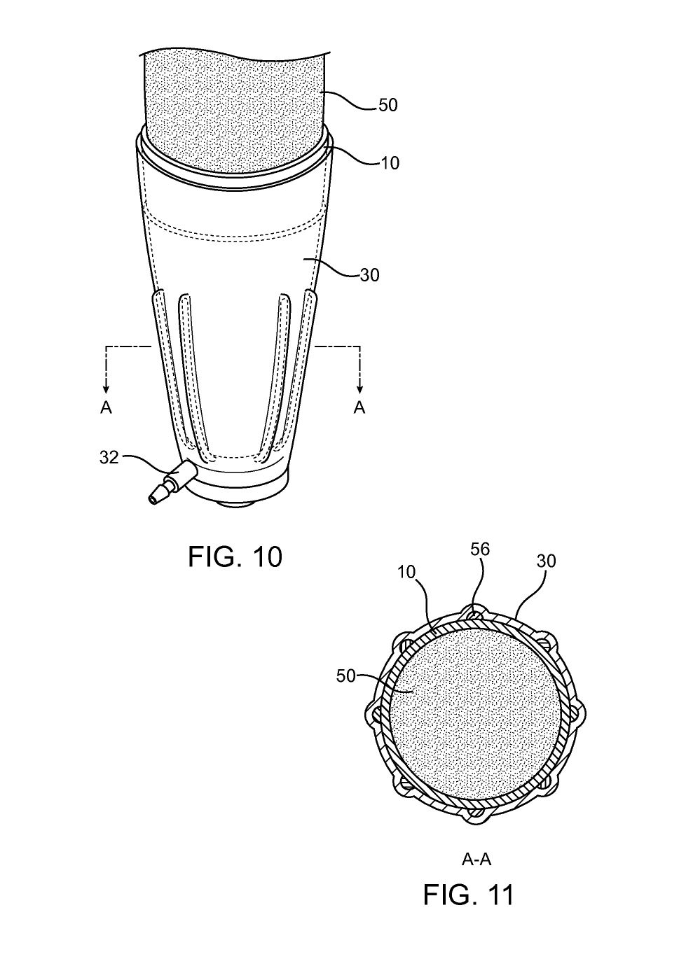

Thereafter, the socket 30 is formed over the liner 10 and tool 52 in a manner known in the art to achieve the assembly shown in FIGS. 10 and 11. The form 50, liner 10 and tool 52 are thereafter removed from socket 30. Liner 10 and socket 30 may then be mounted on the patient's residual limb.

1-20. (canceled)

21. A system for attaching a prosthetic limb to a residual limb, the prosthetic limb having a socket having an interior surface, the system comprising:
an elastic liner adapted to accommodate the residual limb and position within the socket of the prosthetic limb, the elastic liner having an open end, a closed end, an inner surface, and an interior;
an annular groove disposed on the inner surface of the liner; and
a flexible annular seal extending from an inner surface of the liner between the grove and the closed end, the seal extending upwardly toward the open end from the inner surface between the groove and closed end, the seal diverging radially inwardly from the inner surface as it extends upwardly toward the open end, the annular groove being adapted to receive at least part of the annular seal as the annular seal is moved upward and radially outward.

22. The system for attaching a prosthetic limb as recited in claim 21, wherein the liner has an exterior surface and has an an annular element on the exterior surface for engaging the inside surface of the socket.

23. The liner of claim 22, wherein the liner has a vacuum port disposed at the closed end of the liner configured to communicate a vacuum source with the interior of the sleeve.

24. A system for attaching a prosthetic limb to a residual limb, the system comprising:
an elastic liner elastic liner having an open end, a closed end, an inner surface, and an interior adapted for accommodating the residual limb;
an annular groove disposed on the inner surface of the liner; and
a flexible annular seal extending from an inner surface of the liner between the grove and the closed end, the seal upwardly toward the open end from the inner surface between the groove and closed end, the seal diverging radially inwardly from the inner surface as it extends upwardly toward the open end, the annular groove being adapted to receive at least part of the annular seal as the annular seal is moved upward and radially outward;
a socket for attaching to the prosthetic limb, the socket having an interior with a closed end and an open end, the socket receiving the elastic liner in said interior.

25. The system as recited in claim 24, wherein the socket has a vacuum port and an open vacuum channel between the liner and the inner surface of the socket, and a plurality of vacuum channels extending radially outward from the interior surface of the socket.

26. The system as recited in claim 25, wherein the vacuum channels extend longitudinally toward the open end of the socket, the channels being configured to configured enable fluid communication through the vacuum channels between the vacuum port and the interior of the socket when the residual limb and liner are disposed in the socket.
